(19) United States
(12) Patent Application Publication
(10) Pub. No.: US 2017/0326221 A1
(43) Pub. Date: Nov. 16, 2017

(54) Detoxification method for lipopolysaccharide (LPS) or lipid A of Gram-negative bacteria

(71) Applicants: Jean Haensler, Grezieu La Varenne (FR); Francois Dalencon, Lyon (FR); Monique Moreau, Lyon (FR); Noelle Mistretta, Sain Bel (FR)

(72) Inventors: Jean Haensler, Grezieu La Varenne (FR); Francois Dalencon, Lyon (FR); Monique Moreau, Lyon (FR); Noelle Mistretta, Sain Bel (FR)

(73) Assignee: Sanofi Pasteur SA, Lyon (FR)

(21) Appl. No.: 15/584,647

(22) Filed: Aug. 7, 2017

Related U.S. Application Data
(63) Continuation of application No. 12/800,426, filed on May 14, 2010, now abandoned.
(60) Provisional application No. 61/271,985, filed on Jul. 29, 2009.

(30) Foreign Application Priority Data
May 14, 2009 (FR) 0902331

(51) Int. Cl. A61K 39/095; A61K 39/39; A61K 39/00

(52) U.S. Cl. CPC A61K 39/095; A61K 39/39; A61K 2039/55583; A61K 2039/55555

(57) ABSTRACT

The invention relates to a method of detoxifying a lipopolysaccharide (LPS) or a lipid A from a Gram-negative bacterium, which comprises mixing the LPS or the lipid A with a cationic lipid so as to form a complex in which the LPS or the lipid A is associated with the cationic lipid. According to the conventional preparation modes, the cationic lipid with the co-lipid, if this latter is present, get(s) structured into complexes i.a. liposomes. When preparing lipidic complexes, the addition of LPS or Lipid A leads to an association of this latter with the cationic lipid and as a result, the LPS or lipid A is substantially detoxified. The LPS or lipid A detoxified by the complexes, e.g. when incorporated into liposomes, can be used as vaccinal antigen or as adjuvant.

This application is a continuation of U.S. patent application Ser. No. 12/800,426 filed on May 14, 2010, which claims priority to U.S. Provisional Patent Application Ser. No. 61/271,985 filed on Jul. 29, 2009, the disclosures of which are incorporated herein by reference in their entirety.

The invention lies within the vaccine field and relates to a method of detoxifying a lipopolysaccharide (LPS) or a lipid A from a Gram-negative bacterium, which may then be used in vaccine compositions as adjuvant and/or vaccinal antigen.

LPS is a major constituent of the outer membrane of the wall of Gram-negative bacteria. LPS is toxic at high doses to mammals and, in view of this biological activity, has been called an endotoxin. It is responsible for septic shock, a fatal pathology which develops following acute infection with a Gram-negative bacterium.

The structure of LPS is constituted of a lipid portion, called lipid A, covalently bonded to a polysaccharide portion.

Lipid A is responsible for the toxicity of LPS. It is highly hydrophobic and enables the LPS to be anchored in the outer membrane of the wall. Lipid A is composed of a disaccharide structure substituted with fatty acid chains. The number and the composition of the fatty acid chains varies from one species to the other.

The polysaccharide portion is constituted of carbohydrate chains which are responsible for the antigenicity. At least 3 major regions can be distinguished in this polysaccharide portion:

(i) an inner core constituted of monosaccharides [one or more KDO (2-keto-3-deoxyoctulosonic acid) and one or more heptosis (Hep)] which are invariant within the same bacterial species;
(ii) an outer core bonded to heptose and constituted of various monosaccharides; and
(iii) an O-specific outer chain constituted of a series of repeating units—these repeating units themselves being composed of one or more different monosaccharides.

The composition of the polysaccharide portion varies from one species to another, from one serotype (immunotype in meningococcus) to another within the same species.

In a certain number of non-enteric Gram-negative bacteria such as Neisseriae, Bordetellae, Branhamellas, Haemophilus and Moraxellae, the O-specific chain does not exist. The LPS saccharide portion of these bacteria is constituted only of the oligosaccharide core. Consequently, the LPS from these bacteria is often called lipooligosaccharide (LOS).

LPS is not only toxic, it is also immunogenic. In mammals, anti-LPS antibodies are generated during carrying and infection and can be protective. Thus, the use of LPS has already been envisioned in the prophylaxis of infections due to Gram-negative bacteria and associated diseases. Moreover, when it is associated with another antigen of interest, it can also exhibit an adjuvant effect—that is it is able to increase the immune response of a mammal against the associated antigen.

Nevertheless, LPS need to be detoxified before use in vaccinal compositions. To this end, there is no need to remove the entire lipid A. Indeed, the toxic effect being more particularly associated with a supra molecular conformation conferred by the whole lipidic chains borne by the disaccharide core of lipid A, in an advantageous manner, it is sufficient to act at the lipid chain level. Detoxification may be achieved according to various approaches: chemical, enzymatic, genetic or, alternatively by complexation with a polymixin B analogous peptide or by associating the LPS with lipids so as to form complexes such as liposomes. Indeed, the LPS or lipid A in liposomes—that is associated with the lipidic bilayer that constitutes the liposomes—can be substantially detoxified. Lipids complexes i.a. liposomes, for association with/incorporation of LPS or lipid A may be composed of neutral, cationic and/or anionic lipids. This is described in (i) Petrov et al, Infect. Immun. (1992) 60 (9): 3897 which uses a mixture of neutral lipids, phosphatidylcholine and cholesterol; (ii) Richards et al, Vaccine (1989) 7: 506, which uses a mixture of neutral lipids (dimyristoyl phosphatidylcholine, cholesterol) and anionic lipids (dicetyl phosphate, dimyristoyl phosphatidylglycerol); (iii) Bennett-Guerrero et al, Infect. Immun. (2000) 68 (11): 6202, which uses a mixture of neutral lipids (dimysristoyl phosphatidyl choline et cholesterol) et anionique (dimysristoyl phosphatidylglycérol); and Tseng et al, Vet. Immunol. Immunopath. (2009) 131: 285 which in particular uses a mixture of cholesterol, stearyl amine and 1,2-di-palmitoyl-sn-glycero-3-phospho-L-serine (DPPC) leading to the production of cationic liposomes.

Comparatives studies have now shown that cationic lipids exhibit a higher detoxifying power than that of neutral or anionic liposomes. The assays that were achieved for comparison purposes are the followings:
The pyrogenic assay in rabbit. This assay as well the calculation and the reading were achieved according to the European Pharmacopeia Guidelines (Ed 6.0. paragraph 2.6.8.).
The Limulus Amebocyte Lysate (LAL) assay, achieved according to the European Pharmacopeia Guidelines (Ed 6.0. paragraph 2.6.14.).

This is the reason why the invention relates to a method of detoxifying a lipopolysaccharide (LPS) or a lipid A from a Gram-negative bacterium, which comprises mixing the LPS or the lipid A with a cationic lipid so as to form a complex in which the LPS or the lipid A is associated with the cationic lipid.

### BRIEF DESCRIPTION OF THE DRAWINGS

FIG. 1 displays the amount of induced IgG anti-LPS 35 days after the first injection of mice with LPS, as described in section 6.3 (below).

FIG. 2 displays the amount of induced IgM anti-LPS 35 days after the first injection of mice with LPS, as described in section 6.4 (below).

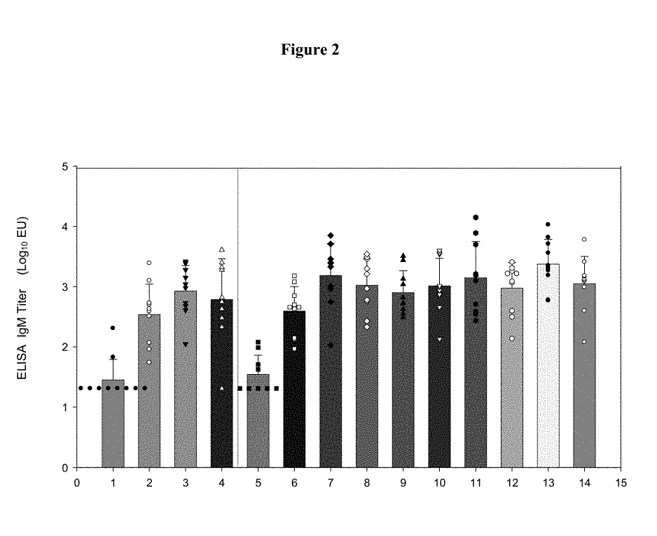

The patent or application file contains at least one drawing executed in color. Copies of this patent or patent application publication with color drawing(s) will be provided by the Office upon request and payment of the necessary fee.

In addition to this the invention also relates to a complex comprising at least LPS or lipid A from a Gram-negative bacterium and a cationic lipid, in which the LPS or the lipid A is detoxified as a result of the complexation thereof with the cationic lipid.

### LPS/Lipid A

The LPS:lipid A that can be detoxified according to the method of the invention may be any LPS of Gram-negative bacteria, whether they are enteric or non-enteric, preferably pathogenic. According to one particular aspect, it may be LPS:lipid A of non-enteric bacteria of genera such as Neisseriae, Bordetellae, Branhamellas, Haemophilus and Moraxellae. The LPS from these bacteria is also referred to as LOS (lipooligosaccharide) owing to the absence of O-specific polysaccharide. By way of additional example, mention is made of LPS/LOS from the genera or species Klebsiella, Pseudomonas, Burkolderia, Porphyromonas, Franciscella, Yersinia, Enterobacter, Salmonella, Shigella or E. coli; and most particularly the LOS from N. meningitidis.

N. meningitidis strains are classified in several immunotypes (IT L1 to IT L13), as a function of their reactivity with a series of antibodies that recognize various LOS epitopes (Achtman et al, 1992, J. Infect. Dis. 165: 53-68). As a direct consequence of this, the LOS from these N. meningitidis strains may also be referred to LOS of immunotype L1 to L13. The differences between immunotypes originate from variations in the composition and in the conformation of the oligosaccharide chains. This is shown in the table below, derived from Table 2 of Braun et al, Vaccine (2004) 22: 898, supplemented with data obtained subsequently and relating to immunotypes L9 (Choudhury et al, Carbohydr. Res. (2008) 343: 2771) and L11 (Mistretta et al, (2008) Poster at the 16th International Pathogenic Neisseria Conference, Rotterdam):

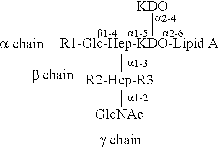

| IT | α chain (including the R1 substituent) | R1 | R2 |
|---|---|---|---|
| L1 | NeuNAcα2-6Galα1-4Galβ1-4Glcβ1-4 | PEA-3 | — |
| L2 | NeuNAcα2-3Galβ1-4GlcNAcβ1-3Galβ1-4 Glcβ1-4 | Glcα (1-3)** | PEA-6 or PEA-7 |
| L3 | NeuNAcα2-3Galβ1-4GlcNAcβ1-3Galβ1-4 Glcβ1-4 | PEA-3 | — |
| L4 | NeuNAcα2-3Galβ1-4GlcNAcβ1-3Galβ1-4 Glcβ1-4 | — | PEA-6 |
| L5 | NeuNAcα2-3Galβ1-4GlcNAcβ1-3Galβ1-4Glcβ1-4Glcβ1-4 | Glcα (1-3) | — |
| L6 | GlcNAcβ1-3Galβ1-4 Glcβ1-4 | — | PEA-6 or PEA-7 |
| L7 | Galβ1-4GlcNAcβ1-3Galβ1-4 Glcβ1-4 | PEA-3 | — |
| L8 | Galβ1-4 Glcβ1-4 | PEA-3 | — |
| L9 | Galβ1-4GlcNAcβ1-3Galβ1-4 Glcβ1-4 | — | PEA-6 |
| L10 | n.d. | n.d. | n.d. |
| L11 | Glcβ1-4Glcβ1-4 | PEA-3 | PEA-6. |
| L12 | n.d | n.d. | n.d. |
| L13 | n.d | n.d. | n.d. |

n.d.: not determined.
**When R2 is a glucose residue, R2 is commonly called β chain.

It may be noted, inter alia, that certain LOSs may be sialylated (presence of N-acetylneuraminic acid on the terminal galactose residue (Gal) of the α chain). Thus, immunotypes L3 and L7 differ only by the respective presence/absence of this sialylation. Moreover, most LOSs are substituted with an O-acetyl group on the glucosamine residue (α-GlcNAc or γ chain) of the inner core (Wakarchuk et al. (1998) Eur. J. Biochem. 254: 626; Gamian et al. (1992) J. Biol. Chem. 267: 922; Kogan et al (1997) Carbohydr. Res. 298: 191; Di Fabio et al. (1990) Can. J. Chem. 68: 1029; Michon et al. (1990) J. Biol. Chem. 275: 9716; Choudhury et al. (above); and Mistretta et al. (above)).

The Galβ1-4GlcNAcβ1-3Galβ1-4Glcβ1-4 carbohydrate motif or lacto-N-neotetraose motif which is present in the α-chain of certain N. meningitidis LPS immunotypes constitutes an epitope which can potentially crossreact with human erythrocytes. Thus, with a view to producing a vaccine for use in humans, it is advisable to choose an LPS which does not possess this unit. It may therefore be particularly advantageous to use an LOS of immunotype L8.

Alternatively, it is also possible to envisage starting, for example, from a strain of immunotype L2 or L3 in which a gene involved in the biosynthesis of the α chain has been inactivated by mutation, so as to obtain an incomplete LNnT structure. Such mutations are proposed in patent application WO 04/014417. This involves extinguishing, by mutation, the lgtB, lgtE (or lgtH), lgtA or lgtA and lgtC genes. Thus, it appears to be possible and advantageous to use an LPS originating from an N. meningitidis strain of immunotype L2 or L3 which is lgtB⁻, lgtE⁻ (or lgtH⁻), lgtA⁻ or lgtA⁻ and lgtC⁻.

For the purposes of the present invention, the LPS may be obtained by conventional means: in particular, it can be extracted from a bacterial culture, and then purified according to conventional methods. Many methods of production are described in the literature. By way of example, mention is made, i.a., of Westphal & Jann, (1965) Meth. Carbohydr. Chem. 5: 83; Gu & Tsai, 1993, Infect. Immun. 61 (5): 1873; Wu et al, 1987, Anal. Biochem. 160: 281 and U.S. Pat. No. 6,531,131. An LPS preparation can be quantified according to well-known procedures. Assaying of KDO by high performance anion exchange chromatography (HPAEC-PAD) is a method which is most particularly suitable.

Turning to lipid A, it may be obtained i.a. by acidic hydrolysis of LPS as described in Gu & Tsai, Infect. Immun. (1993) 61 (5): 1873.

### The Complex

The complex according to the invention or obtained from the detoxifying process of the invention is a cationic complex (positively charged). Typically, it can be a cationic liposome.

By “liposomes” it is meant a synthetic entity, preferably a synthetic vesicle, formed of at least one lipid bilayer membrane (or matrix) enclosing an aqueous compartment. For the purposes of the present invention, the liposomes may be unilamellar (a single bilayer membrane) or multilamellar (several onion-like membranes). The lipids constituting the bi-layer membrane, comprise a non-polar region which, typically, is composed of fatty acid chain(s) or cholesterol, and a polar region, typically composed of a phosphate group and/or tertiary or quaternary ammonium salts. Depending on its composition, the polar region may, in particular at physiological pH (pH≈7) carry either a negative (anionic lipid) or positive (cationic lipid) net (overall) surface charge, or not carry a net charge (neutral lipid).

The complexes i.a. the liposomes, useful in the present invention, can be any type of lipidic complexes exhibiting a global positive charge, i.a. cationic liposomes. The complex is composed of at least one cationic lipid. The cationic lipid can be accompanied with anionic lipids provided the global charge of the complex remains positive.

### The Cationic Lipid

For use in the present invention, the cationic lipid can be:

(i) lipophilic amines or alkylamines such as, for example, dimethyldioctadecylammonium (DDA), trimethyldioctadecylammonium (DTA) or structural homologs of DDA and of DTA [these alkylamines are advantageously used in the form of a salt; mention is made, for example, of dimethyldioctadecylammonium bromide (DDAB)];
(ii) octadecenoyloxy(ethyl-2-heptadecenyl-3-hydroxyethyl)imidazolinium (DOTIM) and structural homologs thereof;
(iii) lipospermines such as N-palmitoyl-D-erythrosphingosyl-1-O-carbamoylspermine (CCS) and dioctadecylamidoglycylspermine (DOGS, transfectam);
(iv) lipids incorporating an ethylphosphocholine structure, such as cationic derivatives of phospholipids, in particular phosphoric ester derivatives of phosphatidylcholine, for example those described in patent application WO 05/049080 and including, in particular:
1,2-dimyristoyl-sn-glycero-3-ethylphosphocholine,
1,2-dipalmitoyl-sn-glycero-3-ethylphosphocholine,
1,2-palmitoyloleoyl-sn-glycero-3-ethylphosphocholine,
1,2-distearoyl-sn-glycero-3-ethylphosphocholine (DSPC),
1,2-dioleyl-sn-glycero-3-ethylphosphocholine (DOEPC or EDOPC or ethyl-DOPC or ethyl PC),
structural homologs thereof; and
   (v) lipids incorporating a trimethylammonium structure, such as N-(1-[2,3-dioleyloxy]propyl)-N,N,N-trimethylammonium (DOTMA) and structural homologs thereof and those incorporating a trimethylammonium propane structure, such as 1,2-dioleyl-3-trimethylammonium propane (DOTAP) and structural homologs thereof; and also lipids incorporating a dimethylammonium structure, such as 1,2-dioleyl-3-dimethylammonium propane (DODAP) and structural homologs thereof; and
   (vi) cationic derivatives of cholesterol, such as 3β-[N—(N′,N′-dimethylaminoethane)-carbamoyl] cholesterol (DC-Chol) or other cationic derivatives of cholesterol, such as those described in U.S. Pat. No. 5,283,185, and in particular cholesteryl-3β-carboxamidoethylenetrimethylammonium iodide, cholesteryl-3β-carboxyamidoethylene-amine, cholesteryl-3β-oxysuccinamidoethylenetrimethylammonium iodide and 3β-[N-(polyethyleneimine)carbamoyl]cholesterol.

By “structural homologs” it is meant lipids which have the characteristic structure of the reference lipid while at the same time differing therefrom by virtue of secondary modifications, especially in the non-polar region, in particular of the number of carbon atoms and of double bonds in the fatty acid chains.

These fatty acids, which are also found in the neutral and anionic phospholipids, are, for example, dodecanoic or lauric acid (C12:0), tetradecanoic or myristic acid (C14:0), hexadecanoic or palmitic acid (C16:0), cis-9-hexadecanoic or palmitoleic acid (C16:1), octadecanoic or stearic acid (C18:0), cis-9-octadecanoic or oleic acid (C18:1), cis,cis-9,12-octadecadienoic or linoleic acid (C18:2), cis-cis-6,9-octadecadienoic acid (C18:2), all-cis-9,12,15-octadecatrienoic or α-linolenic acid (C18:3), all-cis-6,9,12-octadecatrienoic or γ-linolenic acid (C18:3), eicosanoic or arachidic acid (C20:0), cis-9-eicosenoic or gadoleic acid (C20:1), all-cis-8,11,14-eicosatrienoic acid (C20:3), all-cis-5,8,11,14-eicosatetraenoic or arachidonic acid (C20:4), all-cis-5,8,11,14,17-eicosapentaneoic acid (C20:5), docosanoic or behenic acid (C22:0), all-cis-7,10,13,16,19-docosapentaenoic acid (C22:5), all-cis-4,7,10,13,16,19-docosahexaenoic acid (C22:6) and tetracosanoic or lignoceric acid (C24:0).

The characteristic structure of DDAB is:

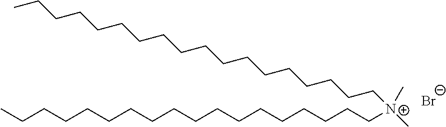 [SMILES: CCCCCCCCCCCCCCCCCC[N+](C)(C)CCCCCCCCCCCCCCCCCC ; [Br-]]

The characteristic structure of ethyl-DOPC is:

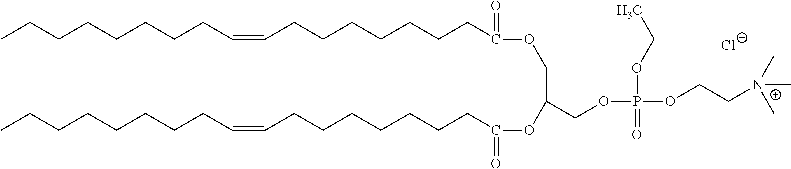 [SMILES: CCCCCCCC/C=C\CCCCCCCC(=O)OCC(COP(=O)(OCC)OCC[N+](C)(C)C)OC(=O)CCCCCCC/C=C\CCCCCCCC ; [Cl-]]

The characteristic structure of DOTAP is:

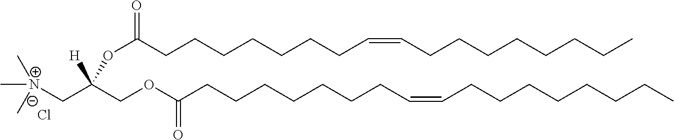 [SMILES: CCCCCCCC/C=C\CCCCCCCC(=O)OC[C@H](C[N+](C)(C)C)OC(=O)CCCCCCC/C=C\CCCCCCCC ; Cl]

The characteristic structure of DC-chol is:

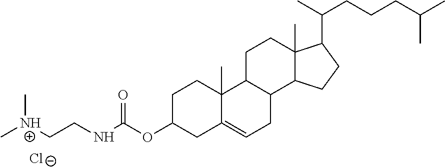 [SMILES: CC(C)CCCC(C)C1CCC2C3CC=C4CC(OC(=O)NCC[NH+](C)C)CCC4(C)C3CCC12C ; [Cl-]]

In a general manner, the cationic lipid can be used in association with a neutral lipid which is often designated under the term “co-lipid”. In an advantageous embodiment, the molar ratio charged lipid (cationic lipid with or without anionic lipid) is from 10:1 to 1:10, advantageously from 4:1 to 1:4, preferably from 3:1 to 1:3.

As a matter of example the following neutral lipids are cited: (i) cholesterol; (ii) phosphatidylcholines such as, for example, 1,2-diacyl-sn-glycero-3-phosphocholines, e.g. 1,2-dioleoyl-sn-glycero-3-phosphocholine (DOPC), and also 1-acyl-2-acyl-sn-glycero-3-phosphocholines of which the acyl chains are different than one another (mixed acyl chains); and (iii) phosphatidylethanolamines such as, for example, 1,2-diacyl-sn-glycero-3-phosphoethanolamines, e.g. 1,2-dioleoyl-sn-glycero-3-phosphoethanolamine (DOPE), and also 1-acyl-2-acyl-sn-glycero-3-phosphoethanolamines bearing mixed acyl chains.

According to one particular embodiment, a mixture of cationic lipid and neutral lipid is used. By way of example, mention is made of:
a mixture of DC-chol and DOPE, in particular in a DC-chol:DOPE molar ratio ranging from 10:1 to 1:10, advantageously from 4:1 to 1:4, preferably from approximately 3:1 to 1:3;
a mixture of EDOPC and cholesterol, in particular in an EDOPC:cholesterol molar ratio ranging from 10:1 to 1:10, advantageously from 4:1 to 1:4, preferably from approximately 3:1 to 1:3; and
a mixture of EDOPC and DOPE, in particular in an EDOPC:DOPE molar ratio ranging from 10:1 to 1:10, advantageously from 4:1 to 1:4, preferably from approximately 3:1 to 1:3.

Several techniques available to the man skilled in the art are useful for preparing liposomes containing LPS (liposomes [LPS]). These different techniques may be more or less appropriate depending on the nature and the properties of LPS, in particular depending on the LPS solubility in aqueous or organic phase. The skilled man is perfectly able to select the most appropriate technique with regard to a particular LPS.

As a matter of example, LPs may be incorporated into liposomes while preparing a dry lipid film which is then rehydrated with a LPS aqueous solution as described in Dijkstra et al, J. Immunol. (1988) 114: 197-205. Alternatively, if LPS is soluble in the organic solvent used for dissolving lipids, it is possible to directly prepare the organic solution containing both the lipids and the LPS which is dried to produce a dry lipid film which is then rehydrated with an aqueous buffer so as to form LPS-containing liposomes. In a general manner, the reconstitution step in aqueous medium leads to the spontaneous formation of multi-lamellar vesicles the size of which is further homogenized while decreasing in a stepwise manner the number of lamellas by extrusion with an extruder under nitrogen pressure, through polycarbonate membranes having smaller and smaller pore diameters (0.8, 0.4, 0.2 μm).

LPS may be also incorporated into liposomes according to the “dehydratation-rehydratation”technique, wherein preformed liposomes are mixed with LPS in aqueous solution, sonicated, lyophilised and dissolved again in an aqueous buffer. This technique is for example used by Petrov et al, Infect. Immun. (1992) 60: 3897.

LPS may be also incorporated into liposomes according to the detergent dilution technique wherein LPS/lipids mixed micelles in detergent are diluted in an aqueous buffer in order to reach a detergent concentration inferior to the detergent critical micellar concentration. At this point, liposomes LPS are formed. This technique is used for example by Argita et al, Vaccine (2005) 23: 5091. This is an equivalent method to that described in the experimental data that follow and illustrate the general description of the invention.

According to one advantageous preparation method, in an initial step, a dry lipid film is prepared with all the compounds that go to make up the composition of the liposomes. The lipid film is then reconstituted in an aqueous medium, in the presence of LPS, for example in a lipid:LPS molar ratio of 100 to 500, advantageously of 100 to 400, preferably of 200 to 300, most particularly preferably of approximately 250. In general, it is considered that this same molar ratio should not substantially vary at the end of the method of preparing the LPS liposomes.

In general, the reconstitution step in an aqueous medium results in the spontaneous formation of multilamellar vesicles, the size of which is subsequently homogenized by gradually decreasing the number of lamellae by extrusion, for example using an extruder, by passing the lipid suspension, under a nitrogen pressure, through polycarbonate membranes with decreasing pore diameters (0.8, 0.4, 0.2 μm). The extrusion process can also be replaced with another process using a detergent (surfactant) which disperses lipids. This detergent is subsequently removed by dialysis or by adsorption onto porous polystyrene microbeads with a particular affinity for detergent (BioBeads). When the surfactant is removed from the lipid dispersion, the lipids reorganize in a double layer.

At the end of the incorporation of the LPS into liposomes, a mixture constituted of ad hoc liposomes and of LPS in free form may commonly be obtained. Advantageously, the liposomes are then purified in order to be rid of the LPS in free form.

Taken into account the LPS/lipid A property, a complex of the invention may be used either as adjuvant in a vaccine composition comprising any king of vaccinal antigen; or as vaccinal antigen in a vaccine composition against infections caused by Gram-negative bacteria; or as adjuvant and vaccinal antigen.

### Vaccines/Method of Treatment

According to another aspect, the invention relates to a vaccine composition which comprises a complex comprising at least LPS or lipid A from a Gram-negative bacterium and a cationic lipid, in which the LPS or the lipid A is detoxified as a result of the complexation thereof with the cationic lipid.

A vaccine composition according to the invention is in particular useful for treating or preventing an infection with a Gram-negative bacterium which is a non-enteric bacterium (such as bacteria of the genera Neisseriae, Bordetellae, Branhamellas, Haemophilus and Moraxellae); or of the genera Klebsiella, Pseudomonas, Burkolderia, Porphyromonas, Franciscella, Yersinia, Enterobacter, Salmonella, Shigella, Escherichia, e.g. E. coli.

According to a preferred aspect, LPS for use in the composition of the invention is the LPS of N. meningitidis and accordingly, the vaccine composition thereof is in particular useful for treating or preventing an infection caused by N. meningitidis, such as meningitis caused by N. meningitidis, meningococcemia and complications which can derive therefrom, such as purpura fulminans and septic shock; and also arthritis and pericarditis caused by N. meningitidis.

The composition of the invention may be conventionally produced. In particular, a therapeutically or prophylactically effective amount of LPS is added to a carrier or diluent.

A vaccine according to the invention may further comprise an adjuvant. According to an advantageous embodiment, the adjuvant is a lipoprotein adjuvant such as the lipidated subunit B (TbpB) of the human transferring receptor of N. meningitidis.

The amounts of LPS per vaccine dose which are sufficient to achieve the abovementioned aims, and which are effective from an immunogenic, prophylactic or therapeutic point of view, depend on certain parameters that include the individual treated (adult, adolescent, child or infant), the route of administration and the administration frequency.

Thus, the amount of LPS per dose which is sufficient to achieve the abovementioned aims is in particular between 5 and 500 μg, advantageously between 10 and 200 μg, preferably between 20 and 100 μg, entirely preferably between 20 and 80 μg or between 20 and 60 μg, limits included.

The term “dose” employed above should be understood to denote a volume of vaccine administered to an individual in one go—i.e. at T time. Conventional doses are of the order of a milliliter, for example 0.5, 1 or 1.5 ml; the definitive choice depending on certain parameters, and in particular on the age and the status of the recipient. An individual can receive a dose divided up into injections at several injection sites on the same day. The dose may be a single dose or, if necessary, the individual may also receive several doses a certain time apart—it being possible for this time apart to be determined by those skilled in the art.

The composition of the invention may be administered by any conventional route in use in the art, e.g. in the vaccines field, in particular enterally or parenterally. The administration may be carried out as a single dose or as repeated doses a certain time apart. The route of administration varies as a function of various parameters, for example of the individual treated (condition, age, etc.).

Finally, the invention also relates to:
a method of inducing in a mammal, for example a human, an immune response against a Gram-negative pathogenic bacterium, according to which an immunogenically effective amount of a vaccine according to the invention is administered to the mammal so as to induce an immune response, in particular a protective immune response against the Gram-negative pathogenic bacterium; and
a method for prevention and/or treatment of an infection caused by a Gram-negative pathogenic bacterium, according to which a prophylactically or therapeutically effective amount of a vaccine according to the invention is administered to an individual in need of such a treatment.

The invention is illustrated by the experimental section as follows.

### Experimental Data

### 1. Purified LPS Preparation

### Culture

Eight ml of frozen sample of an N. meningitidis serotype A strain known to exclusively express LPS immunotype L8 are used to inoculate 800 ml of Mueller-Hinton medium (Merck) supplemented with 4 ml of a solution of glucose at 500 g/l and divided up in Erlenmeyer flasks. The culture is continued with shaking at 36±1° C. for approximately 10 hours.

400 ml of a solution of glucose at 500 g/l and 800 ml of a solution of amino acids are added to the preculture. This preparation is used to inoculate a fermentor containing Mueller-Hinton medium, at an OD₆₀₀ₙₘ close to 0.05. The fermentation is continued at 36° C., at pH 6.8, 100 rpm, pO₂ 30% under an initial airstream of 0.75 l/min/l of culture.

After approximately 7 hours (OD₆₀₀ₙₘ of approximately 3), Mueller-Hinton medium is added at a rate of 440 g/h. When the glucose concentration is less than 5 g/l, the fermentation is stopped. The final OD₆₀₀ₙₘ is commonly between 20 and 40. The cells are harvested by centrifugation and the pellets are frozen at −35° C.

### Purification

The pellets are thawed and suspended with 3 volumes of 4.5% (vol./vol.) phenol with vigorous stirring for 4 hours at approximately 5° C. The LPS is extracted by phenol treatment.

The bacterial suspension is heated to 65° C. and then mixed vol./vol. with 90% phenol, with vigorous stirring for 50-70 min at 65° C. The suspension is subsequently cooled to ambient temperature and then centrifuged for 20 min at 11 000 g. The aqueous phase is removed and stored, while the phenolic phase and the interphase are harvested so as to be subjected to a second extraction.

The phenolic phase and the interphase are heated to 65° C. and then mixed with a volume of water equivalent to that of the aqueous phase previously removed, with vigorous stirring for 50-70 min at 65° C. The suspension is subsequently cooled to ambient temperature and then centrifuged for 20 min at 11 000 g. The aqueous phase is removed and stored, while the phenolic phase and the interphase are harvested so as to be subjected to a third extraction identical to the second.

The three aqueous phases are dialyzed separately, each against 40 μl of water. The dialysates are then combined. One volume of 20 mM Tris, 2 mM MgCl₂ is added to 9 volumes of dialysate. The pH is adjusted to 8.0±0.2 with 4N sodium hydroxide.

Two hundred and fifty international units of DNAse are added per gram of pellet. The pH is adjusted to 6.8±0.2. The preparation is placed at 37° C. for approximately 2 hours with magnetic stirring, and then subjected to filtration through a 0.22 μm membrane. The filtrate is purified by passing it through a SEPHACRYL® S-300 column (5.0×90 cm; PHARMACIA™).

The fractions containing the LPS are combined and the MgCl₂ concentration is increased to 0.5M by adding powdered MgCl₂.6H₂O, with stirring.

While continuing the stirring, dehydrated absolute alcohol is added to give a final concentration of 55% (vol./vol.). The stirring is continued overnight at 5±2° C., and then centrifugation is carried at 5000 g for 30 min at 5±2° C. The pellets are resuspended with at least 100 ml of 0.5M MgCl₂ and then subjected to a second alcoholic precipitation identical to the preceding one. The pellets are resuspended with at least 100 ml of 0.5M MgCl₂.

The suspension is subjected to a gel filtration as previously described. The fractions containing the LPS are combined and filtration-sterilized (0.8-0.22 μm) and stored at 5±2° C.

This purification method makes it possible to obtain approximately 150 mg of LPS L8 per liter of culture.

### 2. Preparation of LPS Liposomes (i.a., Lipids: EDOPC and DOPE)

### 2.1. Production of Liposomes [LPS L8] by Detergent Dialysis

The LPS L8 liposomes are prepared by detergent dialysis. Briefly, the lipids (EDOPC:DOPE) prepared as a lipid film and taken up in 10 mM Tris buffer, and then dispersed in the presence of 100 mM of octyl-β-D-glucopyranoside (OG) (Sigma-Aldrich ref. 08001) and filtered under sterile conditions. The LPS L8 in 100 mM OG is added under sterile conditions. The lipids/LPS/OG mixture is then dialyzed against 10 mM Tris buffer in order to remove the OG and form liposomes.

### Protocol

A lipid preparation in chloroform, of the lipids that will be used to produce the liposomes, is prepared. A dry film is obtained by complete evaporation of the chloroform.

A dry film of 1,2-dioleoyl-sn-glycero-3-ethylphosphocholine (EDOPC or ethyl-DOPC) and of 1,2-dioleoyl-sn-glycero-3-phosphoethanolamine (DOPE) in an EDOPC:DOPE molar ratio of 3 to 2 is obtained by mixing 12.633 ml of a solution of EDOPC (Avanti Polar Lipids ref. 890704) at 20 mg/ml in chloroform and 7.367 ml of a solution of DOPE (Avanti Polar Lipids ref. 850725) at 20 mg/ml in chloroform, and evaporating off the chloroform until it has completely disappeared.

The dry film is taken up with 30 ml of 10 mM Tris buffer, pH 7.0, so as to obtain a suspension containing 13.333 mg of lipids/ml (8.42 mg/ml of EDOPC and 4.91 mg/ml of DOPE). The suspension is stirred for 1 hour at ambient temperature and then sonicated for 5 min in a bath.

3.333 ml of a sterile 1M solution of octyl-β-D-glucopyranoside (OG) (Sigma-Aldrich ref. O8001) in 10 mM Tris buffer, pH 7.0, are then added, still with stirring, so as to obtain a clear suspension of lipids at 12 mg/ml, 100 mM OG and 10 mM Tris buffer. The stirring is continued for 1 h at ambient temperature on a platform shaker. Filtration is then carried out sterilely through a Millex HV 0.45 μm filter.

A composition is prepared, under sterile conditions, by mixing together LPS and lipids in a lipids:LPS molar ratio of 250 (0.160 mg/ml of LPS L8, 9.412 mg/ml of lipids and 100 mM of OG). 40 ml of such a composition are obtained from mixing the following preparations:

2.005 ml of 10 mM Tris buffer, pH 7.0; 0.223 ml of 100 mM OG in 10 mM Tris; 31.373 ml of the EDOPC:DOPE suspension having a molar ratio of 3:2, at 12 mg/ml in 100 mM OG, 10 mM Tris; and 6.4 ml of a sterile suspension of LPS L8 at 1 mg/ml in 100 mM OG, 10 mM Tris.

After stirring for one hour at ambient temperature, the suspension is transferred under sterile conditions into 4 sterile 10 ml dialysis cassettes. Each cassette is dialyzed 3 times (24 hrs-24 hrs-72 hrs) against 200 volumes of 10 mM Tris, pH 7.0, i.e. 2 l.

The liposomes are recovered under sterile conditions. The increase in volume after dialysis is approximately 30%.

Merthiolate and NaCl are added to this preparation so as to obtain a preparation of liposomes in 10 mM Tris, 150 mM NaCl, pH 7.0, 0.001% merthiolate, which ultimately contains approximately 110 μg/ml of LPS and 7 mg/ml of lipids, of which there are approximately 4.5 mg/ml of EDOPC and approximately 2.5 mg/ml of DOPE (theoretical concentrations).

The LPS liposomes are stored at +5° C.

### 2.2. Production of Liposomes [LPS L8] by Extrusion

Liposomes [LPS L8] are prepared with DC-chol or EDOPC in a lipid/LPs molar ratio of 250.

To this end, 129 μg of LPS and 5.2 mg of DC-Chol or 10.4 mg of EDOPC are dissolved in 10 mL of a mixture chloroforme/methanol 4:1. A dry film is obtained while evaporating the solvent and complementary drying under vacuum. The dry film is taken up with ultra-filtered water at 50° C. and stirred. The preparation is sonicated then submitted to extrusion upon a single membrane filtration (retention threshold: 0.4 μm) followed by six membrane filtration ((retention threshold: 0.2 μm). The preparation is finally sterilized by filtration.

### 3. Evaluation of the LPS Detoxification.

Three main assays are used: (i) The LAL (Limulus Amebocyte Lysate) assay; (ii) the IL6 and TNFα cytokines in vitro release assay; and (iii) the rabbit pyrogen assay.

### LAL Assay

The LAL assay is a colorimetric assay which is very sensitive and allowing the detection and quantification of endotoxins of Gram-negative bacteria. This assay is achieved according to the European Pharmacopeia guidelines (Edition 5.0., paragraph 2.6.14.) using the QCL-1000 kit ref; 50-647 U de CAMBREX-BIOWHITAKER™ (linear zone: 0.1 à 1 UI/mL) with as a negative control, the E. coli endotoxin, 4·10³ EU/mL (SIGMA™).

Samples to be tested as well as the standard and the positive control are diluted in the respective ranges of 1/10 à 1/10⁵; 0.5 à 0.031 EU/mL; et 1/10⁴ à 1.8 10⁴.

Fifty μL of the dilutions of the samples, standard and positive control are distributed in wells of a 96-well ELISA plate. 50 μL of lysate are added to each well; then 100 μL of p-nitroaniline are added as well. The plate is incubated 6 min a 37° C. The reaction is stopped while adding 100 μL of frozen acetic acid (25% in water). The plate is read by spectrometry at 405 nm.

Evaluation of the endotoxin concentration: The mean value of the optical density (OD) of the <<white>> sample is substracted from the test sample OD. The linear regression curve of the standard range is drawn up (it must be linear from 0.031 EU/mL to 0.5 EU/mL) in order to evaluate the endotoxin concentration (EU/mL) of each test sample starting with the read ODs. Then these values are multiplied by the reverse of the corresponding dilutions and the mean arithmetic value is calculated.

The detoxification rate is determined as being the LAL value measured with non-formulated LPS divided by the LAL value measured with the product formulated in liposomes provided that the LPS concentration in both samples is equivalent.

### Cytokine In Vitro Release Assay

Human blood in natrium heparin (25,000 U/5 mL; Sanofi Aventis) is diluted to the fifth in AIM-V medium (Invitrogen™). 400 μL per well of this preparation is distributed in MICRONICS™ tubes. 100 μL of the substances to be tested are added. The tubes are incubated 24 hrs à 37° C. under a wet atmosphere loaded with 5% CO₂.

Tubes are centrifuged 10 min at 500 g. From each tube at least 200 μL of plasmatic supernatant are recovered and kept frozen at −80° C. until the dosage is achieved.

The cytokine dosage is achieved by ELISA with the OptEIA IL6, human IL8 and TNFα kits from Pharmingen™, each of the kits comprising a capture antibody (mouse anti-human cytokine antibody), a detection antibody (mouse biotinylated anti-human cytokine antibody), an avidine-peroxydase conjugate and standards.

The capture antibodies are diluted to 1/250 in carbonate buffer 0.1 M pH 9.5 (Sigma™). For each assay, 100 μL of the 1/250 dilution are distributed in each well of a 96-well ELISA plate (Maxisorp NUNC 96™). The plates are incubated overnight at 4° C.

Plates are washed in PBS 0.05% Tween 20. 200 μL of PBS 0.05% bovine sérumalbumine are added per well. The plates are incubated 1 hr at room temperature. The plates are washed with PBS 0.05% Tween 20.

Dilutions of recombinant cytokines in AIM-V medium are prepared in the following range: 1,200 pg/mL-18.75 pg/mL (IL6); 800 pg/mL-12.5 pg/mL (IL8); et 1,000 pg/mL-15.87 pg/mL (TNFα). 100 μL of each dilution are distributed in wells in order to establish the standard curve.

Plasmas recovered from blood stimulated with pure LPS are diluted to 1/250 and 1/125. Those recovered from blood in touch with liposomes LPS are diluted to 1/5 and 1/25. 100 μL of each dilution are distributed per well. Plates are incubated 2 hrs at room temperature.

Plates are washed with PBS 0.05% Tween 20. The detection antibody and the enzyme are both diluted to 1/250 in PBS containing 10% de fetal calf serum. 100 μl of each dilution are added per well. Plates are incubated one hour at room temperature.

Plates are washed with PBS 0.05% Tween 20. 100 μl of substrate are distributed per well (tetramethylbenzidine solutions A et B mixed vol. à vol). Plates are incubated 10 to 30 min at room temperature.

The reaction is stopped by adding per well, 100 μL of phosphoric acid 1 M. Plates are read at 450 nm.

Standard curves for cytokine concentration as a function of optical density are obtained from a recombinant cytokine dilution range, and the rough results correspond to the sample concentration read on these standard curves.

The detoxification rate is determined as being the ratio of the concentration of liposome-formulated LPS that induces 50% of maximum release (ED50 expressed in pg/mL) divided by the concentration of non-formulated LPS that induces 50% of maximum cytokine release. The higher the rate, the higher the detoxification. Since the detoxification rate is systematically measured while using blood from several independents donors, the results express a mean value.

### Rabbit Pyrogen Assay

Rabbit is considered as being the animal having a sensitivity to the LPS pyrogenic effects equivalent to that observed in humans. The pyrogen assay consists in measuring the temperature increase induced by an intravenous injection of a sterile solution of the substances to be analyzed. The assay, reading and calculations thereof are achieved according to the European Pharmacopeia guidelines (Edition 6.0, paragraph 2.6.8.). A pyrogenic effect is recorded when the temperature increase is over 1.15° C.

### 4. Mice Immunogenicity Study

### Mouse Immunisation

Seven-week old CD1 female mice (Charles River Lab.) distributed in several groups receive by the sub-cutaneous route, 200 μl of preparations containing 50 μg/mL LPS in Tris 10 mM NaCl 150 mM pH 7.0. Blood samples are recovered before each of the injections Mice are sacrified at D35.

### Anti-LPS Antibody Dosage by ELISA

The ELISA dosage of LPS specific antibodies in the serum samples was performed by a robotic application (Staccato robot, Caliper) according to the following protocols:

Dynex 96-well microplates were coated with 1 μg of L8 LPS, in phosphate buffered saline (PBS) 1× pH 7.1+MgCl₂ 10 mM. microplates are incubated 2 hours at +37° C. and then overnight at +4° C. Plates were then blocked for 1 hour at 37° C. with 150 μl of PBS-0.05% Tween 20-1% (w/v) powdered skim milk. All subsequent incubations were carried out in a final volume of 100 μl, followed by 3 washings with PBS-0.05% Tween 20.

Serial two-fold dilutions of the samples performed in PBS-Tween-milk (starting by 1/40), were added to the wells and incubated for 90 min at 37° C. After washings 3 times, an anti-rabbit or anti-mouse IgG (1/10 000) peroxidase conjugate diluted in PBS-Tween-milk was added and the plates incubated for another 90 min at 37° C. The plates were further washed (3 times) and incubated in the dark for 20 min at room temperature with 100 μl per well of a ready-to-use TMB substrate solution (TMB: 3,3′,5,5′-tétraméthylbenzidine, peroxidase substrate). The reactions were stopped with 100 μl of 1 M HCl.

The optical density (OD) was measured at 450-650 nm with an automatic plate reader (Multiskan Ascent). As no standard is available, the antibodies titers were determined as the reciprocal dilution giving an OD of 1.0 on a curve plotted with the two values that border the OD of 1. The threshold of antibody detection was of 1.3 log₁₀ ELISA units (EU). For each titer inferior to this threshold, an arbitrary vale of 1.3 log₁₀ was assigned.

### 5. LPS and Lipid Quantification

### 5.1. Dosage of Lipids by HPLC-UV

### Preparation of the Standard Range and of the Samples to be Analyzed

A stock solution containing 1 mg/ml, in chloroform, of each of the EDOPC and DOPE lipids is prepared and is subsequently diluted to 1/10^{th} by adding an acetonitrile/water (90/10) mixture. This stock solution is used to prepare the standard range of 2 to 50 μl/ml by dilution in acetonitrile/water mixture.

The samples to be analyzed are diluted in acetonitrile/water so as to have a theoretical final concentration of about 10 μg/ml.

### Analytical Conditions

A Zorbax C18 Extend, 3; 5 μm, 3×150 mm, 80A column (Agilent reference 763954-302) is used, and for the mobile phase, an acetonitrile/water/trifluoroacetic acid (TFA) mixture in the volume proportions 850/150/1 is used. The column is pre-conditioned according to the following process:
flow rate at 0.25 ml/min for 20 minutes (P=21 bar)
flow rate at 0.5 ml/min for 20 minutes (P=42 bar)
flow rate at 0.75 ml/min for 20 minutes (P=60 bar)
flow rate at 1 ml/min for 20 minutes (P=80 bar)

The measurements are carried out at 60° C., by injecting 10 μl of the preparation at a mobile-phase flow rate of 1 ml/min. The analytes are detected at OD 200 nm.

DC-chol average retention time: 1.6 minutes
EDOPC average retention time: 7.7 minutes
DOPC average retention time: 9.9 minutes
DOPE average retention time: 11.5 minutes
Cholesterol average retention time: 13.4 minutes

### 5.2. Dosage of LPS by HPAEC-PAD

The principle of the assay consists in submitting LPS to an acid hydrolysis which releases one molecule of KDO per molecule of LPS; then in separating this free KDO from the rest and in quantifying it by high performance ion exchange chromatography with pulsed amperometric detection (HPAEC-PAD).

### Preparation of Standard Range and Analytes

The following are prepared in a final volume of 400 μl: a blank and a standard range of KDO of between 42.5 and 1700 ng/ml; which corresponds to a LPS standard range from 613 to 24507 ng of LPS/ml. The blank and each of the samples of the range also contain an amount of lipids and/or of detergent substantially equivalent to that present in the samples to be assayed; that is to say, e.g. 0.7 mg/ml of a mixture of EDOPC and of DOPE in a molar ratio of 3:2 together with 0.2 mM octyl glucoside.

The samples to be assayed are prepared under a final volume of 400 μl by dilution, e.g. to 1/10^{th}, of a liposomes preparation at starting theoretical LPS concentration of 100 μg/ml.

### Acid Hydrolysis

100 μl of a hydrolysis solution containing 5% acetic acid and glucuronic acid at 20 μg/ml (compound used as internal standard) prepared extemporaneously are introduced into the standard range+blank samples and into the samples to be assayed. The hydrolysis is allowed to continue for 1 h at 100° C. and is then stopped by centrifugation at 5° C. for 5 min.

### Extraction of the Lipids and the Detergent

500 μl of purified water are added to the hydrolysis product, followed by 2 ml of a 2/1 mixture of chloroform/methanol, and the mixture is vortexed for 30 sec. It is centrifuged at 4500 rpm for 10 min. The aqueous phases are taken, dried at 45° C. for 2 hours under a nitrogen stream at 0.5 bar and taken up with 400 μl of water.

### HPAEC-PAD Assay

This technique is implemented on an HPAEC system (DIONEX™) using the DIONEX™ CHROMELEON® management software for the data acquisition and reprocessing. The chromatography column (CARBOPAC® PA1×250 mm (DIONEX™ reference 035391)) is subjected to a temperature of 30° C. The column is equilibrated with an eluting solution (75 mM NaOH, 90 mM NaOAc) and pre-conditioned according to the following scheme:
flow rate at 0.20 ml/min for 20 minutes (P=270 psi)
flow rate at 0.4 ml/min for 20 minutes (P=540 psi)
flow rate at 0.6 ml/min for 20 minutes (P=800 psi)
flow rate at 0.8 ml/min for 20 minutes (P=1055 psi)
flow rate at 1 ml/min for 20 minutes (P=1300 psi)

100 μl of a sample are injected onto the column at an elution flow rate of 1 ml/min for 22 min.

The amount of KDO present in the sample is determined by integration of the KDO peak of the chromatogram. Since one mole of KDO is released per mole of LPS, it is possible to determine the concentration of LPS present in the initial preparation.

### 6. Results

6.1. Cationic Liposomes have Superior Detoxifying Property

Three kinds of LPS-containing liposomes have been prepared according to the extrusion method: (i) liposomes containing a single lipid, this latter being a neutral lipid (DOPC); (ii) liposomes containing a single lipid, this latter being a cationic lipid (EDOPC or DC-chol; and (iii) liposomes containing a cationic lipid and a neutral lipid. These liposomes are described in the following table which also shows the results of the LAL and pyrogen assay. LPS incorporated into neutral liposomes induce a pyrogenic effect in rabbit at LPS amounts which do not mediate this effect when LPS is incorporated into cationic liposomes.

| | Amount of product | | | | | |
|---|---|---|---|---|---|---|
| | (LPS, lipid 1, lipid 2) | | | | | |
| | used for preparing liposomes | | | | LAL EU | |
| | | | | Molar | (mean from | Pyrogen |
| | LPS | Lipid 1 | Lipid 2 | ratio | 3 assays)/ | assay |
| Lipid 1:Lipid 2 | (μg/ml) | (μg/mL) | (μg/mL) | lipids:LPS | μg de LPS | (rabbit) |
| DC-chol | 9.92 | 400 | | 250 | 5080 | Non- |
| | | | | | (detoxifying | pyrogenic at |
| | | | | | factor: 16) | 10, 25 and |
| EDOPC:DOPE | 9.92 | 381 | 223 | | 824 | 50 ng/ml |
| | | | | | (detoxifying | LPS |
| | | | | | factor: 35) | |
| EDOPC | 9.92 | 635 | | | 12791 | |
| | | | | | (detoxifying | |
| | | | | | factor: 2) | |
| DC-chol:DOPE | 9.92 | 201 | 222 | | 5335 | |
| DOPC | 9.92 | 585 | | | | Pyrogenic |
| (neutral) | | | | | | at 25 and 50 ng/ml |
| | | | | | | LPS |
| LPS | 10 | | | | 28656 | |
| Not treated | | | | | | |

6.2. LPS Detoxification is Studied as a Function of the Lipid:LPS Molar Ratio, of the Liposome Composition and/or the Liposome Preparation Process

Various kinds of LPS-containing liposomes have been prepared either by extrusion or by detergent dialysis. Their composition is described in the following table. The size of liposomes is analyzed by quasi-elastic light diffusion using a Malvern Zetasizer nano-S apparatus. The size measured is largely inferior to 200 nm.

| | | | Theorical values | | Actual concentrations | | | |
|---|---|---|---|---|---|---|---|---|
| | | | (before process) | | (after process) | | | |
| | | | Molar | | | | | DC-chol |
| | Lipids | | ratio | LPS | LPS | EDOPC | DOPE | ou Chol |
| | (mol/mol) | Technique | Lipid/LPS | (μg/mL) | (μg/mL) | (μg/mL) | (μg/mL) | (μg/mL) |
| A | EDOPC:DOPE | OG dialysis | | 100 | 81 | 215 | 90 | |
| B | (3:2) | | 100 | | 78 | 1334 | 570 | |
| C | | | 175 | | 82 | 1796 | 980 | |
| D | | | 250 | | 85 | 2678 | 1580 | |
| E | | | 325 | | 78 | 3365 | 2280 | |
| F | | | 400 | | 80 | 4148 | 2890 | |
| Fbis | | | 400 | | 86 | 3650 | 2680 | |
| G | EDOPC:chol. | OG dialysis | 250 | | 64 | 2960 | | 530 |
| | (7:3) | | | | | | | |
| H | DC-chol | Extrusion | 250 | | 34 | | | 3080 |
| I | EDOPC | Extrusion | 250 | | 63 | 5151 | | |
| J | EDOPC:DOPE | OG dialysis | — | 0 | | 3695 | 241 | |
| | (3:2) | | | | | | | |

LPS detoxification is measured at TO and T+3 months with the LAL and cytokine assays.

The results are to be shown in the following table:

| | | | | Detoxification | | | |
|---|---|---|---|---|---|---|---|
| | | | | IL6 release | | LAL | |
| | Lipids | | Molar ratio | | T = | | T = |
| | (mol/mol) | Technique | Lipid/LPS | T = 0 | +3 mois | T = 0 | +3 mois |
| A | EDOPC:DOPE | OG dialysis | 20 | 2198 | 3252 | 4 | 43 |
| B | (3:2) | | 100 | 751 | 2796 | 11 | 148 |
| C | | | 175 | 1229 | 6430 | 33 | 236 |
| D | | | 250 | 1089 | 1574 | 163 | 270 |
| E | | | 325 | 860 | 1170 | 321 | 187 |
| F | | | 400 | 648 | 754 | 360 | 310 |
| G | EDOPC:chol. | OG dialysis | 250 | 279 | 2359 | 166 | 157 |
| | (7:3) | | | | | | |
| H | DC-chol | Extrusion | 250 | 144 | 258 | 6 | 4 |
| I | EDOPC | Extrusion | 250 | 23 | 271 | 38 | 154 |
| J | EDOPC:DOPE | OG dialysis | — | No IL6 | No IL6 | Nd | Nd |
| | (3:2) | | | secretion | secretion | | |

| | | | | |
|---|---|---|---|---|
| Purified LPS | 1 | 1 | 1 | 1 |
| Endotoxoid | 49 | 65 | 2178 | Nd |

Nd: Not determined.

The detoxification rate measured by IL6 release is determined as being the ratio of the concentration of LPS formulated in liposomes which induces 50% of maximum release (ED50 expressed in pg/mL)/the concentration of non-formulated LPS which induces 50% of maximum release.

In the LAL assay, the detoxification rate is expressed as being the LAL value measured with the non-formulated LPS divided by the LAL value measured with the product formulated in liposomes, the LPS concentration being equivalent.

Even if the detoxification rates seem to follow the LPS concentration in liposomes (both assays showing inverse tendencies), it is not possible to conclude that there are substantial differences from a concentration to another. The LPS concentration in liposomes should not have any incidence on detoxification. By contrast, addition of a co-lipid (cholesterol or DOPE) to the cationic lipid (EDOPC) seems to be beneficial to detoxification.

The detoxification of LPS in liposomes is also compared to that obtained with the endotoxoid obtained by complexation of the purified LPS with the SAEP2-L2 peptide (dimeric, anti-parallel) following the instruction given in WO 06/108586. The detoxification is not as high as those observed with the endotoxoid; but still perfectly acceptable since the SAEP2-L2 peptide detoxifies LPS beyond what is required.

6.3. LPS Immunogenicity is Studied as a Function of the Lipid: LPS Molar Ratio, of the Liposome Composition and/or the Liposome Preparation Process

Groups of 10 mice are constituted. Mice were immunized at D0 and D21 with 10 μg of LPS with an injection of an aliquot of preparations A-I of liposomes LPS at 50 μg/ml in Tris buffer 10 mM, NaCl 150 mM, pH 7.0. Negative and positive controls are added to the test. The positive controls are the following: purified, non-detoxified LPS from the same batch (10 μg par injection) as the one used to prepare the liposomes LPS as well as 10 μg LPS from this bath in an endotoxoid form—endotoxoid prepared according to WO 06/108586.

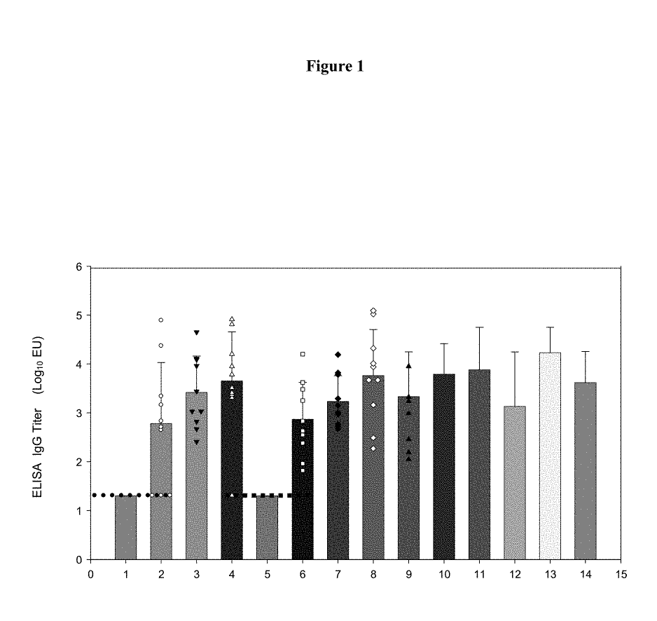

The amounts of IgG and IgM anti-LPS induced have been evaluated by ELISA 35 days after the first injection. The results are to be shown in FIGS. 1 (IgG) and 2 (IgM). In each of these figures, numbers 6 to 14 and 5 respectively correspond to samples A à J described in the tables hereinabove. Sample 1 is a control sample solely constituted by a buffer solution. Sample 3 contains non-detoxified LPS L8. Samples 2 and 4 are references samples containing the endotoxoid (LPS L8 detoxified upon complexation with peptide SAEP2 L2, which is a polymixine B analog)

Whatever the LPS formulation mode, the antigenic character of detoxified LPS exhibits extensive homogeneity. LPS formulated in liposomes is able to induce antibodies as non-detoxified LPS and endotoxoid do.

1. A method of detoxifying a lipopolysaccharide (LPS) or a lipid A from a Gram-negative bacterium, which comprises
mixing the LPS or the lipid A with at least a cationic lipid so as to form a net positively charged liposome in which the LPS or the lipid A is complexed with the cationic lipid in the liposome bilayer,
wherein
the mixing and liposome formation of the LPS or the lipid A with the cationic lipid results in detoxification of the LPS or lipid A,
and wherein
the cationic lipid is a lipid incorporating an ethylphosphocholine structure or a cationic derivative of cholesterol.

2. The method as claimed in claim 1, wherein the LPS is a lipooligosaccharide (LOS) from Neisseria meningitidis.

3. The method as claimed in claim 1, wherein the cationic lipid is 1,2-dioleoyl-sn-glycero-3-ethylphosphocholine (DOEPC or EDOPC) or 3β-[N—(N′,N′-dimethylaminoethane) carbamoyl]cholesterol (DC-Chol).

4. The method as claimed in claim 1, wherein a neutral lipid (colipid) is mixed with the cationic lipid and the LPS or the lipid A so as to form a liposome incorporating the LPS or the lipid A.

5. The method as claimed in claim 4, wherein the neutral lipid is selected from the group constituted of (i) cholesterol; (ii) phosphatidylcholines; and (iii) phosphatidylethanolamines.

6. The method as claimed in claim 5, wherein the neutral lipid is 1,2-dioleoyl-sn-glycero-3-phosphoethanolamine (DOPE).

7. A composition comprising at least LPS or lipid A from a Gram-negative bacterium and at least a cationic lipid, wherein the composition comprises a net positively charged liposome incorporating LPS or lipid A from a Gram-negative bacterium in the liposome bilayer, wherein the LPS or lipid A is complexed with the cationic lipid, and wherein the LPS or the lipid A is detoxified as a result of incorporation in the liposome, and wherein the cationic lipid is a lipid incorporating an ethylphosphocholine structure or a cationic derivative of cholesterol.

8. The composition as claimed in claim 7, wherein the LPS is a lipooligosaccharide from Neisseria meningitidis.

9. The composition as claimed in claim 7, in which the cationic lipid is EDOPC or DC-Chol.

10. The composition as claimed in claim 7, which additionally comprises a neutral lipid.

11. The composition as claimed in claim 10, wherein the neutral lipid is selected from the group constituted of (i) cholesterol; (ii) phosphatidylcholines; and (iii) phosphatidylethanolamines.

12. The composition as claimed in claim 11, wherein the neutral lipid is DOPE.

13. A method of adjuvanting an antigen which comprises mixing the antigen with a composition as claimed in claim 7.

14. An immunogenic composition comprising a composition as claimed in claim 7, optionally in combination with a lipoprotein adjuvant.

15. A method of inducing an immune response in an individual against a lipopolysaccharide or a lipid A from a Gram-negative bacterium, the method comprising administering to the individual a composition according to claim 7.

16. The method of claim 15, wherein the LPS is a lipooligosaccharide from Neisseria meningitidis.
